(19) United States
(12) Patent Application Publication
(10) Pub. No.: US 2010/0047115 A1
(43) Pub. Date: Feb. 25, 2010

(54) METHOD OF AND APPARATUS FOR CLEANING AND DISINFECTION OF AIR

(75) Inventors: Igor A. Krichtafovitch, Kirkland, WA (US); Gergey Karpov, Chestnut Hill, MA (US)

Correspondence Address: MORRISON & FOERSTER LLP, 1650 TYSONS BOULEVARD, SUITE 400, MCLEAN, VA, 22102, US

(73) Assignee: Kronos Advanced Technologies, Inc., Massachusetts (US)

(21) Appl. No.: 12/513,973

(22) PCT Filed: Nov. 9, 2007
(86) PCT No.: PCT/US07/84363

§ 371 Date: May 7, 2009

Related U.S. Application Data
(60) Provisional application No. 60/865,130, filed on Nov. 9, 2006.

(51) Int. Cl. A61L 9/00; B01D 50/00; B03C 3/00; B01D 46/00

(52) U.S. Cl. 422/4; 55/385.2; 95/273; 96/15

(57) ABSTRACT

The methods and apparatus according to embodiments of the present invention provide substantially complete and rapid air cleaning and/or disinfection in the rooms that are substantially closed to outside air and have the typical complement and arrangement of furniture and people present, i.e. a “normal”environment. A study of air flow patterns in a variety of room configurations and arrangements demonstrates that EFA location within the room as well as furniture and people play a crucial role in air movement and airflow throughout the room, in minimizing or eliminating “dead spots” of stagnant air, and in achieving a goal of cleaning and disinfecting substantially all of the air.

### CROSS-REFERENCE TO RELATED APPLICATIONS

This application claims the priority of U.S. Provisional Patent Application Ser. No. 60/865,130, filed Nov. 9, 2006, entitled “Method of and Apparatus for Cleaning and Disinfection of Air”, the entire disclosure of which is specifically incorporated herein in its entirety by reference.

### BACKGROUND OF THE INVENTION

1. Field of the Invention

The invention relates to air cleaning and, in particular, to air disinfection in closed premises such as hospital wards and habitats.

2. Description of the Related Art

There are numerous devices and methods of air cleaning and disinfecting that include dragging air or forcing air under pressure through an air cleaning and/or disinfecting device (hereinafter—air cleaner) and discharging clean air back into a closed environment such as a room. Some of these devices use air filtration mechanism such as HEPA filters. Other devices use bacteria or other pathogen deactivating technology such as ultraviolet lamps. Related information may be found in the disclosures of the following patents that are incorporated herein in their entireties by reference:

| Pat. No. | Title |
|---|---|
| US patent | Corona-discharge air mover and purifier for fireplace |
| application | and hearth. |
| 20060112955 | |
| 7,276,106 | Electrode wire retaining member for an electrostatic |
| | precipitator. |
| 7,269,008 | Colling apparatus and method. |
| 7,226,497 | Fanless building ventilator. |
| 7,226,496 | Spot ventilators and method for spot ventilating |
| | bathrooms, kitchens and closets. |
| 7,220,295 | Electrode self-cleaning mechanisms with anti-arc guard |
| | for electro-kinetic air transporter-conditioner devices. |
| 7,182,805 | Corona-discharge air mover and purifier for packaged |
| | terminal and room air conditioners. |
| 7,112,232 | Air cleaning apparatus |
| 7,108,731 | Air cleaning robot and system thereof |
| 7,094,142 | Portable device with interchangeable air cleaning |
| | modules for cleaning the air outside of an existing |
| | enclosed space and delivering the cleaned air into the |
| | existing enclosed space |
| 7,056,368 | Powered air cleaning system and air cleaning method |
| 7,049,569 | Microwave oven with air cleaning function |
| 7,025,812 | Portable air cleaning apparatus |
| 7,025,806 | Electrically enhanced air filtration with improved |
| | efficacy |
| 7,013,656 | Vehicle ventilation and deodorization system |
| 7,010,900 | Beverage bottling plant for filling bottles with a liquid |
| | beverage filling material, and a cleaning device for |
| | cleaning bottles in a beverage bottling plant |
| 6,991,722 | Hydrate desalination for water purification |
| 6,955,152 | Manually operated tool |
| 6,938,626 | Method and apparatus for wet-cleaning substrate |
| 6,929,684 | Air cleaning apparatus |
| 6,926,762 | Air cleaning apparatus |
| 6,913,214 | Powder bell purge tube |
| 6,913,012 | Cooking appliance venting system |
| 6,909,025 | Method and equipment for pre-treatment of used tires |
| | before a pyrolysis process |
| 6,899,668 | Airborne pathogen isolation system and method |
| 6,897,420 | Cooker having air cleaning unit |
| 6,893,618 | Device for air cleaning from dust and aerosols |
| 6,893,340 | Rotary accelerating apparatus for a vertical straw and |
| | chaff spreader of an agricultural combine |
| 6,869,458 | Bioclean room unit |
| 6,863,594 | Method and device for cleaning high-voltage carrying |
| | installation component parts |
| 6,855,295 | UV air cleaning and disinfecting system |
| 6,843,969 | Air cleaning |
| 6,843,835 | Air cleaning apparatus and method for cleaning air |
| 6,830,682 | Controlled cooling of input water by dissociation of |
| | hydrate in an artificially pressurized assisted desalination |
| | fractionation apparatus |
| 6,821,320 | Dust collector for collecting fine dust in air |
| 6,811,593 | Air-cleaning device and method for arranging air |
| | cleaning in sensitive environments |
| 6,808,545 | Portable exhaust fan |
| 6,800,025 | Combine air system for cleanout |
| 6,799,589 | Method and apparatus for wet-cleaning substrate |
| 6,780,213 | Personal air cleaning apparatus |
| 6,777,355 | Manufacturing apparatus and manufacturing method for |
| | semiconductor device |
| 6,769,979 | Automotive air cleaning system |
| 6,767,471 | Hydrate desalination or water purification |
| 6,746,519 | Air cleaning filter |
| 6,739,073 | Method and apparatus for performing multiple cleaning |
| | and vacuum drying operations in enclosed vessels |
| 6,723,146 | Blower apparatus for vehicle |
| 6,712,687 | Big game gambrel |
| 6,702,879 | Air filtering material for air cleaning |
| 6,670,290 | Manufacturing apparatus and manufacturing method for |
| | semiconductor device |
| 6,669,759 | Method for regenerating activated carbon and device for |
| | carrying out said method |
| 6,666,912 | Air cleaning apparatus |
| 6,662,600 | Foamed cleaning liquid dispensing system |
| 6,648,935 | Dual stage extraction blower for removing contaminants |
| | from an air stream |
| 6,584,993 | Portable-type cleaning device for internal combustion |
| | engine |
| 6,544,320 | Air-cleaning apparatus |
| 6,508,982 | Air-cleaning apparatus and air-cleaning method |
| 6,503,462 | Smart air cleaning system and method thereof |
| 6,491,883 | Air-cleaning photocatalytic filter |
| 6,490,754 | Low pressure air cleaning system |
| 6,475,460 | Desalination and concomitant carbon dioxide capture |
| | yielding liquid carbon dioxide |
| 6,462,935 | Replaceable flow-through capacitors for removing |
| | charged species from liquids |
| 6,457,186 | Air cleaning device for a toilet bowl |
| 6,454,834 | Regenerable air cleaning device |
| 6,432,872 | Composition for use in adsorption treatment, products |
| | formed with the same, and a method for producing |
| | adsorbent using the same |
| 6,413,303 | Activated carbon air filters |
| 6,401,730 | Container wash system |
| 6,398,852 | Device for air cleaning |
| 6,378,535 | Hollow article cleaning apparatus and hollow article |
| | cleaning method |
| 6,361,574 | Intake air cleaning apparatus |
| 6,306,190 | Secondary air supply device |
| 6,296,823 | Method and installation for eliminating gaseous organic |
| | substances in the air |
| 6,296,692 | Air purifier |
| 6,293,120 | Building air conditioning system using geothermal |
| | energy |
| 6,287,023 | Processing apparatus and method |
| 6,282,910 | Indoor blower variable speed drive for reduced airflow |
| 6,273,109 | Cleaning device for automobile bodies |
| 6,251,151 | Air cleaner having a curved guide surface for airflow to |
| | an air cleaning element |
| 6,245,131 | Electrostatic air cleaner |
| 6,213,121 | Nasal filtration system |
| 6,194,346 | Photocatalyst and method of making |
| 6,193,772 | Self-propelled harvesting machine having a selectively |
| | engageable suction cleaning device of a filter |
| 6,193,075 | Air classification of animal by-products |
| 6,178,977 | Device for cleaning deposits from an internal combustion |
| | engine |
| 6,178,765 | Air conditioner having air cleaning function |
| 6,168,140 | Air treating device |
| 6,167,862 | Air cleaner system |
| 6,164,082 | Air conditioner with air cleaner |
| 6,151,903 | Air conditioning apparatus and air conditioning method |
| | for reducing electric power consumption by reducing |
| | pressure loss in circulation air |
| 6,146,451 | Air-cleaning filter, method of producing the same, and |
| | high-level cleaning device |
| 6,136,074 | Air conditioning apparatus with an air cleaning function |
| | and electric dust collector for use in the same |
| 6,129,781 | Air conditioning apparatus with an air cleaning function |
| | and electric dust collector for use in the same |
| 6,120,584 | Air cleaning apparatus, air filter and method for |
| | manufacturing the same |
| 6,093,097 | Methods and apparatus for controlling an air inlet closure |
| | of an air conditioner |
| 6,092,424 | Pressure sensor for a baghouse |
| 6,074,981 | Photocatalyst and process for the preparation thereof |
| 6,062,977 | Source capture air filtering device |
| 6,056,797 | Dust collector filter cleaning control system |
| 6,047,049 | Multi-function audio system and method for controlling |
| | the same |
| 6,036,755 | Water filtering type air cleaning unit |
| 6,024,678 | Vacuum cleaner leg exercise device |
| 6,006,471 | Air-cleaning ecosystem apparatus |
| 6,004,382 | Air cleaning system for vehicle cooling system and |
| | engine and cab |
| 5,980,614 | Air cleaning apparatus |
| 5,974,976 | Cleaning system and process for making same employing |
| | reduced air cleaning fabric |
| 5,968,214 | Air cleaning apparatus for vehicles |
| 5,961,829 | Method and an apparatus for the purification of water, |
| | more particularly from a composting process |
| 5,958,112 | Air-cleaning apparatus |
| 5,944,987 | Multipurpose combinatory oil, air, gas, & pollution |
| | filtration system |
| 5,914,414 | Air cleaner |
| 5,909,339 | Method and apparatus for cleaning air in a hard disk |
| | drive |
| 5,901,459 | Shuttle mechanism for twin tower air dryer system |
| 5,887,797 | Bag house cleaning systems |

One of the most efficient technologies to both deactivate, e.g. kill, bacteria and viruses, and simultaneously collect the residual toxic and/or poisonous remnants uses Electrostatic Fluid Accelerators (EFA) to kill pathogens with an electrical field and collect their remnants on electrically charged collecting plates. These air cleaners are developed by Kronos Air Technologies, Inc. It may be found that the geometry, materials, circuit diagrams used for embodiments of the present invention are disclosed in the prior patent applications of Igor Krichtafovitch et al. including

| | | Pat. No./ | |
|---|---|---|---|
| Ser. No. | Filing Date | Publication | Title |
| 09/419,720 | Oct. 14, 1999 | 6,504,308 | Electrostatic Fluid Accelerator |
| 10/175,947 | Jun. 21, 2002 | 6,664,741 | Method Of And Apparatus For Electrostatic |
| | | | Fluid Acceleration Control Of A Fluid Flow |
| 10/188,069 | Jul. 3, 2002 | 6,727,657 | Electrostatic Fluid Accelerator For And A |
| | | | Method Of Controlling Fluid Flow |
| 10/295,869 | Nov. 18, 2002 | 6,888,314 | Electrostatic Fluid Accelerator |
| 10/352,193 | Jan. 28, 2003 | 6,919,698 | Electrostatic Fluid Accelerator For And Method |
| | | | Of Controlling A Fluid Flow |
| 10/187,983 | Jul. 3, 2002 | 6,937,455 | Spark Management Method And Device |
| 10/735,302 | Dec. 15, 2003 | 6,963,479 | Method Of And Apparatus For Electrostatic |
| | | | Fluid Acceleration Control Of A Fluid Flow |
| 10/847,438 | May 18, 2004 | 7,053,565 | Electrostatic Fluid Accelerator For And A |
| | | | Method Of Controlling Fluid Flow |
| 11/210,773 | Aug. 25, 2005 | 7,122,070 | Method Of And Apparatus For Electrostatic |
| | | | Fluid Acceleration Control Of A Fluid Flow |
| 10/806,473 | Mar. 23, 2004 | 20040217720 | Electrostatic Fluid Accelerator For And A |
| | | | Method Of Controlling Fluid Flow |
| 10/724,707 | Dec. 2, 2003 | 20050116166 | Corona Discharge Electrode And Method Of |
| | | | Operating The Same |
| 10/752,530 | Jan. 8, 2004 | 20050150384 | Electrostatic Air Cleaning Device |
| 11/046,711 | Feb. 1, 2005 | 20050151490 | Electrostatic Fluid Accelerator For And Method |
| | | | Of Controlling A Fluid Flow |
| 11/119,748 | May 3, 2005 | 20050200289 | Electrostatic Fluid Accelerator |
| 11/214,066 | Aug. 30, 2005 | 20060055343 | Spark Management Method And Device |
| 11/347,565 | Feb. 6, 2006 | 20060226787 | Electrostatic Fluid Accelerator For And Method |
| | | | Of Controlling A Fluid Flow |

all of which are incorporated herein in their entireties by reference.

Such air cleaners based of Electrostatic Fluid Accelerators, i.e. EFA, may produce a relatively weak (e.g., low pressure) airflow unlike the pressure differential produced by, for example, typical industrial or home vacuum cleaners or powerful (e.g., industrial or home) motor-driven fans, or fan based HEPA air purifiers. Therefore, conventional EFAs may not necessarily be well suited or capable of stirring all of the air present in a room or other enclosed space so as to completely circulate all of the air through the EFA and/or an associated air cleaner within a desired precocity (e.g., four times per hour, etc.) As a result, some air in the room remains undisturbed and, therefore, is not processed, e.g., disinfected or cleaned.

To effectively disinfect a space, an important factor is to properly and suitable direct the air flow of both the contaminated air as it moves toward an intake or inlet port and the cleaned, disinfected air as it is exhausted back into the room. Otherwise, contaminated air (i.e., “infected air”) may transfer and spread germs (e.g., viruses, spores, bacteria, or other disease vectors) from infected and/or contagious persons to a healthy or otherwise uninfected person. This is particularly a problem for those having a weak immune system or otherwise susceptible to infection by the airborne vector thereby having adverse health consequences for the victim.

### SUMMARY OF THE INVENTION

The methods and apparatus according to embodiments of the present invention provide substantially complete and rapid air cleaning and/or disinfection in the rooms that are substantially closed to outside air and have the typical complement and arrangement of furniture and people present, i.e. a “normal” environment. A study of air flow patterns in a variety of room configurations and arrangements demonstrates that EFA location within the room as well as furniture and people play a crucial role in air movement and airflow throughout the room, in minimizing or eliminating “dead spots” of stagnant air, and in achieving a goal of cleaning and disinfecting substantially all of the air.

According to one aspect of the invention, a method of air cleaning and disinfecting, that includes air acceleration by a first means such as an electrostatic fluid accelerator, fan, blower or other device for propelling and/or imparting an acceleration and increasing a speed of the air. An air cleaning and/or disinfection by passing through a second means such as filters, ultraviolet lamps, electrostatic precipitators and the like that remove particulates, destroy pathogens, bacteria, etc., and otherwise increase the safety of the air for humans. An air delivery to an inlet of the second means is performed by the first means.

According to a feature of the invention, a third means provides an air pass configuration that ensures the most of the air is routed to go to the inlet of the second means. This third means may be, for example, a wall, furniture, or other objects that function to route, channel, provide a passage, deflect, conduct, steer or otherwise direct an airflow.

According to another feature of the invention, an air pass configuration by the first means ensures a maximum amount of the air goes to the inlet of the second means.

According to another feature of the invention, at least a portion of the first means is combined with some portion of the second means.

According to another feature of the invention, an air pass is configured in such a way that the air passes through areas of major concern on the way to the inlet of the second means.

According to another feature of the invention, an air pass is configured in such a way that air passes to areas of major concern on the way from an outlet of the second means

According to another feature of the invention, an air pass is configured in such a way that it passes through areas of major concern on the way from an outlet of the first means to an inlet of the first means.

According to another feature of the invention, air is directed by the first means substantially orthogonally to a most distant third means.

According to another feature of the invention, an air pass from the first means to the third means is the longest available in the premises to be cleaned.

According to another feature of the invention, an air pass from an outlet of the first means to the inlet of the second means is shorter than an air pass from an outlet of the first means to an outlet of the second means.

According to another feature of the invention, the first means includes air acceleration by a heat source.

According to another feature of the invention, the first means include air acceleration by an existing ventilation and/or air exchange means.

According to another feature of the invention, the heat source includes one or more of the following: thermal batteries, space heaters, fireplaces, heat exchanges and such.

According to another feature of the invention, air is reflected from the third means in substantially symmetrical fashion.

According to another feature of the invention, air acceleration rates by the third means change with time.

According to another aspect of the invention, a system for processing room air includes an air propelling device located within the room, the air propelling device having an air intake operating to receive ambient room air, an air accelerator for accelerating the room air received by the intake, and an air exhaust operating to expel back into the room air from the air accelerator; an air cleaning device operating to remove material from the room air; and an air delivery device for routing the room air from the air cleaning device to the air intake of the air propelling device.

According to a feature of the invention, the air propelling device comprises an electrostatic fluid accelerator.

According to another feature of the invention, the air propelling device comprises an electric blower or fan.

According to another feature of the invention, the air cleaning device operates to disinfect the room air.

According to another feature of the invention, the air cleaning device comprises one or more of an air filter, ultraviolet lamp and electrostatic precipitator.

According to another feature of the invention, an air deflector directs a stream of air from the outlet of the air propelling device to the air cleaning device and ensures that a major portion of air expelled by the air propelling device arrives at the air cleaning device.

According to another feature of the invention, the air deflector comprises a structural portion of the room.

According to another feature of the invention, the structural portion of the room comprises a wall of the room.

According to another feature of the invention, the air deflector is located at an opposite portion of the room across from the air propelling device.

Additional objects, advantages and novel features of the invention will be set forth in part in the description which follows, and in part will become apparent to those skilled in the art upon examination of the following and the accompanying drawings or may be learned by practice of the invention. The objects and advantages of the invention may be realized and attained by means of the instrumentalities and combinations particularly pointed out in the appended claims.

### BRIEF DESCRIPTION OF THE DRAWINGS

The drawing figures depict preferred embodiments of the present invention by way of example, not by way of limitations. In the figures, like reference numerals refer to the same or similar elements.

FIG. 1a is an overhead plan of a room including placement of an Electronic Fluid Accelerator (EFA) at a window end of the room with various furnishings together with the resultant air flow pattern according to an embodiment of the invention;

FIG. 1b is an overhead plan of a room including placement of an EFA on an inside wall of a room having various furnishings together with the resultant air flow pattern according to another embodiment of the invention;

FIG. 2 is a perspective view of a room including placement of an EFA at a ceiling end of a wall with various room furnishings present together with the resultant air flow pattern according to another embodiment of the invention;

FIG. 3 is a perspective view of a room including placement of an EFA one a table or other support within the room, the room having various furnishings present together with the resultant air flow pattern according to another embodiment of the invention;

FIG. 4 is a diagram depicting various operational modes of an EFA including periodic and/or controlled power and/or airflow rate levels that may be implemented according to various embodiments of the invention;

FIG. 5 is a side view of a room including ceiling placement of an EFA wherein room wall surfaces do not readily support the reflection of airflow such that airflow is instead directed downward toward and reflected by a floor surface;

FIG. 6 is side view of a room including ceiling placement of an EFA wherein room wall and floor surfaces do not readily support the reflection of airflow stash that exhaust airflow is instead directed outward along the ceiling;

FIG. 7 represent an experimental observation of the air flow with a different EFA placement in the room;

FIGS. 8-12 are a computer generated air flow models for one EFA located within a room operated at different settings; and

FIGS. 13-17 are at computer generated air flow models for multiple EFA configurations within a room operated at the different settings.

### DETAILED DESCRIPTION OF THE INVENTION

The ensuing description provides exemplary embodiments only, and is not intended to limit the scope, applicability, or configuration of the invention. Rather, the ensuing description of the exemplary embodiments will provide those skilled in the art with an enabling description for implementing, an example embodiment of the invention. It should be understood that various changes may be made in the function and arrangement of elements without departing from the spirit and scope of the invention.

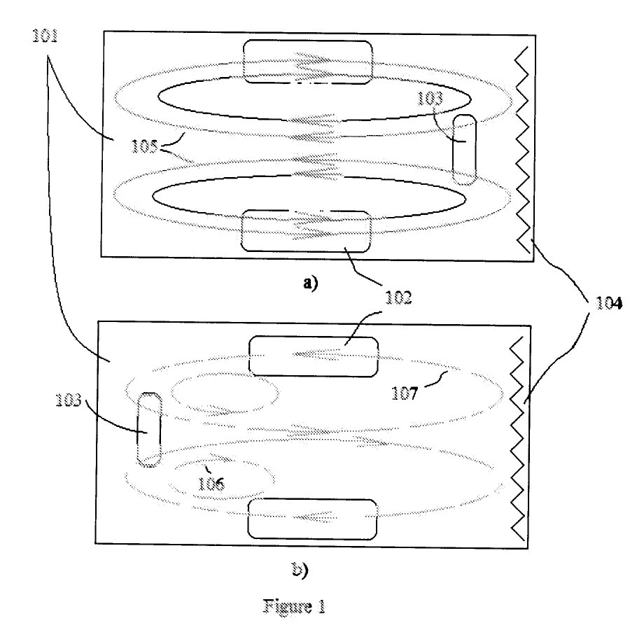

Referring to FIG. 1a, room 101 is substantially rectangular in cross area, containing two patient beds 102 and blinds, curtains, or other mechanism that covers window 104. EFA 103 is placed next to window 104 and is configured to blow air in a direction away from window 104 along path 105. The clean air from EFA 103 reaches the opposite wall, reflects from the opposite wall and returns to EFA 103 along the walls. As a result, substantially all of the air in the room is moved and goes though the EFA where it is processed and cleaned.

FIG. 1b illustrates a similar configuration to that of FIG. 1a except that EFA 103 is located and installed in a symmetrical location, i.e. at the opposite wall, i.e. to the blinds 104. The blinds or curtains have a lower air reflecting ability than the opposite clear wall providing a hard surface to reflect air impinging upon it. Instead the blinds or curtains have the effect of changing the airflow pattern. In this configuration, an air flow pattern is quite different with a predominant direction 107 and two patterns of vortexes. However, now the patient beds 102 are not in the air flow pattern so that there is little airflow (i.e., the air is stagnant in the vicinity of the beds). Therefore, much less cleaning and disinfecting action is performed and more time is needed to clean and disinfect the air. This is because, at least in part, the window blinds or curtains do not effectively reflect but rather avert air flow and do not help to redirect the air back to an air intake of EFA 103. Thus, in light of the advantages of the configuration illustrated by FIG. 1a, a preferred method according to an embodiment of the invention is to provide air cleaning by positioning an EFA or other air cleaning apparatus such that it blows air against a surface that substantially reflects and redirects airflow rather than forcing air to go in local vortexes. Preferably, such surface is substantially hard and flat such as conventional room wall.

In an alternate configuration as illustrated by FIG. 2, a room 201 contains two patient beds 203 and a piece of furniture that avert air flow 204. It was found experimentally that EFA 202 accelerates air in a substantially linear or straight direction, exiting the EFA outlet or exhaust as shown with upper arrows along respective path 205 and 206. This air direction is characteristic for the EFA air cleaner. Thus, according to another embodiment of the invention, a second method of air cleaning prescribes that an EFA air cleaner be location at a predetermined or specified elevation or angle so the accelerated air direction goes beyond (in FIG. 2—above) any obstruction that is likely to avert or weaken reflected air flow.

A third configuration and method of placement of an EFA according to all embodiment of the invention positions the EFA air cleaner between the vulnerable (i.e., uninfected persons who may be the targets or victims of airborne vectors such as viruses, bacteria, spores, contaminants, etc.) and invulnerable people (i.e., infected individuals who may be hosts or sources of infectious agents) such that clean (i.e. disinfected) air goes from invulnerable (e.g., infected) people to the vulnerable (e.g., uninfected) people through the EFA air cleaner.

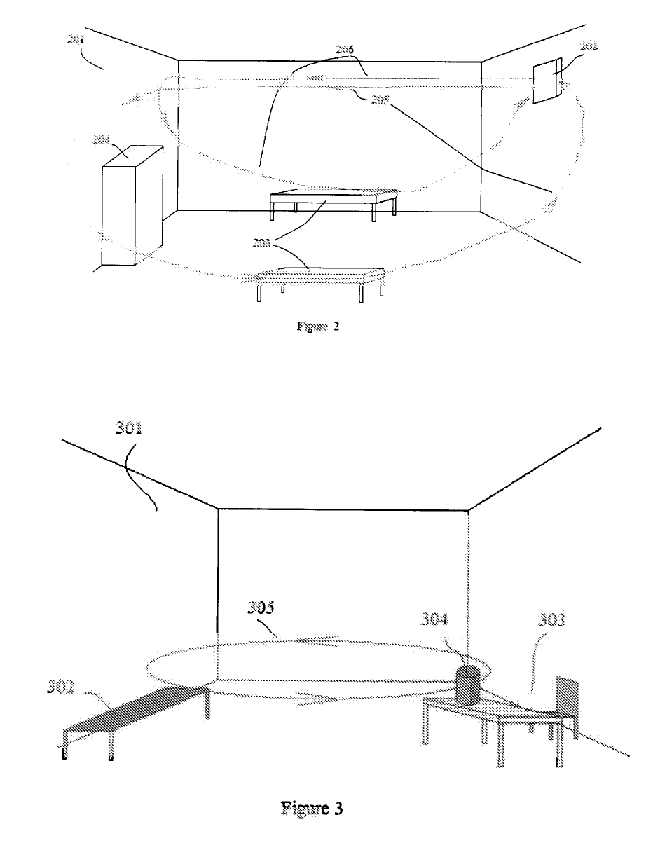

In FIG. 3 a room 301 is shown that contains a patient's bed 302, nurse's desk 303 and EFA air cleaner 304. EFA cleaner 304 is located in such a way that air from the infected patient goes (i.e., is routed) to the healthy nurse (vulnerable to the infection) through EFA 304. In this way cleaned and disinfected air goes to the nurse thus decreasing the risk of her/his infection by airborne agents otherwise imparted into the air by an infected patient. In a case wherein the patient has a weakened immune system (e.g., a newborn or AIDS victim) or is otherwise prone to infection by airborne vectors (e.g., burn and wound victims, patients recently undergoing and recovering from surgery, etc.) such person becomes “vulnerable” while relatively healthy staff personnel (such as physicians, nurses, technicians, etc. who may be germs carriers themselves) are less vulnerable to infection. In such cases the EFA should be position and configured to blow clean and disinfected air toward the patient (who may be intolerant of infectious agents) and away from the staff.

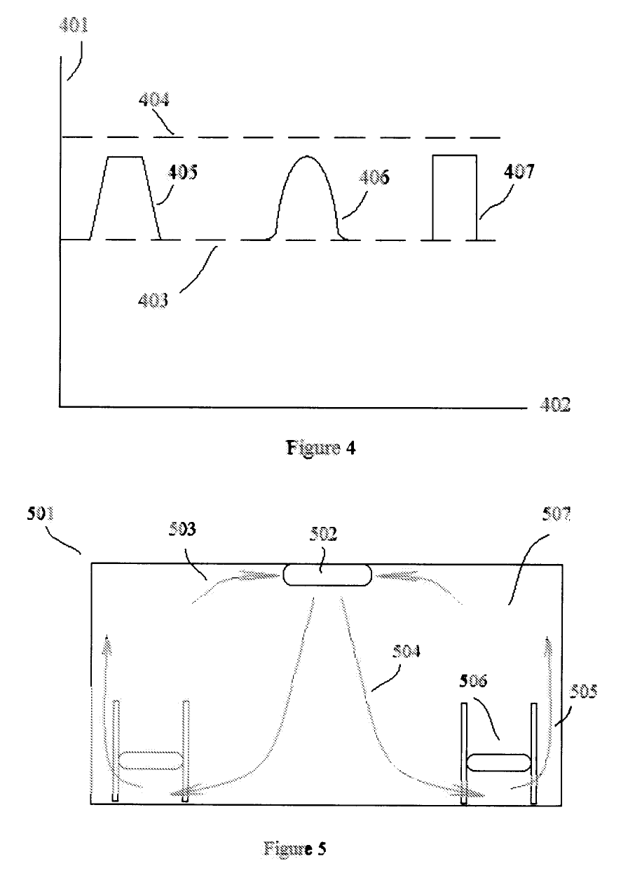

In relatively large rooms and/or rooms with a substantial amount of furniture or other bulky objects, typical EFAs may not produce sufficient air flow to stir air in every corner of the room, i.e., some air may remain stagnant and uncirculated. This is because many EFAs do not normally develop full power under many circumstances and conditions and, instead, operate at some lower power or airflow level of reduced air delivery capacity. For instance, some EFA produce certain amount of ozone and have to operate at lower than maximum capacity. To overcome this deficiency, and EFA may be configured to operate during a substantial portion of time at some low level that is not less than the lowest efficient level of air cleaning and disinfection. This lowest level (for example, an operating level determined by a parameter such as a potential difference applied between the corona and collecting electrodes or as reflected by a desired air flow velocity) is shown in the FIG. 4 as 403 (401 is the voltage or air velocity and 402 is time). From time to time, periodically or as needed (e.g., in response to some measured parameter such as air quality, particulate content, detection of a contagious airborne agent, etc.), EFA capacity is increased for some short time interval so as to move (stir) more air from remote or hidden parts of the room so as to circulate the air and eventually cause it to be brought to the air intake of and be processed by the EFA intake. Thus, implementations of this method may prescribe periodic increases of the EFA capacity in response to time or increased air contamination/infection. This increase may be periodic or non-periodic, and implemented as a power and/or airflow rate that linearly changes over time such as depicted by trapezoid 405; abrupt changes as represented by rectangular area 407; or varies by increasing and decreasing according to some other function as represented by bell-like waveform 406 or any other shape, time or duty cycle.

Embodiments of the invention further address operation in enclosures such as rooms lacking any flat air-reflecting or flat spot on the walls such that it might be problematic to find an appropriate place for an EFA to be installed. In such cases EFA 502 may be installed on a ceiling and positioned to blow air downward toward and onto an unobstructed portion of floor as shown in the FIG. 5. As depicted therein, room 507 is furnished with two beds 506 and EFA 502 that takes air in from opposing sides of EFA 502 flowing along adjacent portions of the ceiling 503. EFA 502 has a downwardly positioned exhaust port configured to discharge clean air toward floor 504. Deflected by floor 504, the air proceeds in along path 505 via patients' beds 506 to recirculate upwardly along sidewalls of the room to the ceiling and back to EFA 502.

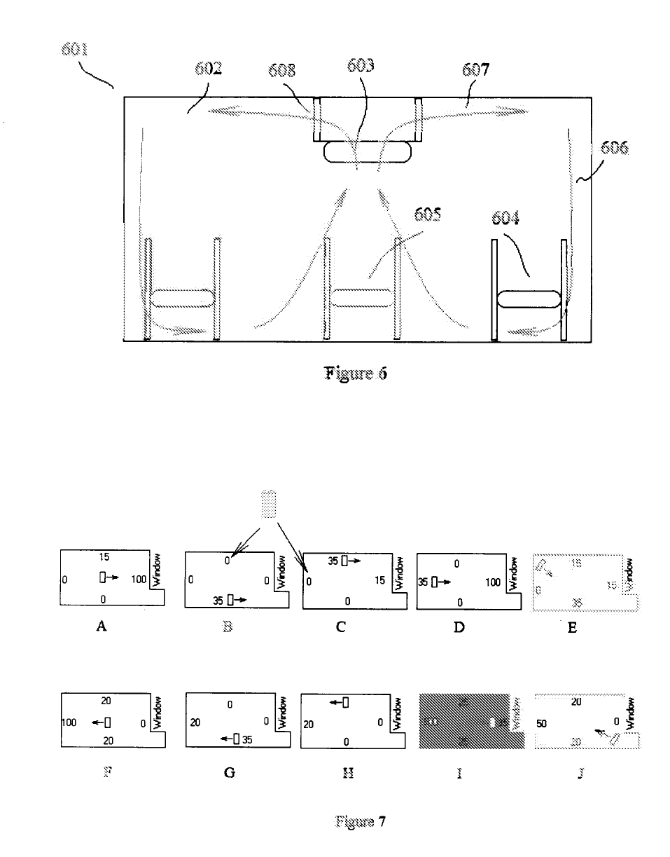

In rooms wherein the floor is not flat (e.g., covered with furniture or other objects) another location supporting efficient operation of an EFA functioning as an air cleaner is shown in the FIG. 6. Therein room 602 contains a number of beds (three are shown for purposes of ease of illustration although any number may be included) with EFA air cleaner 603 located close to the ceiling but spaced some distance from it (e.g., 2 to 8 inches). EFA 603 is supported by standoffs that do not prevent or substantially impede clean air from being directed outward from a top-facing exhaust vent outwardly along ceiling 607. The clean air then follows the arrows 606 downward along sidewalls of the room, along portions of the floor, and then back upward as depicted by arrow 605 to return to an air intake of EFA 603.

In rooms that are substantially rectangular in shape and/or configuration, an EFA air cleaner may be located in three principal directions. If the EFA blows air to the longest path it is more efficient to install it close to the short wall at the center of the short wall, as shown in the FIG. 1a. In this configuration the EFA air cleaner should be located close to the wall. The distance from the wall to the EFA intake should be large enough as to provide an area of air intake that is no less that outlet area.

FIG. 7 shows the result of airflow observation in the room of specific geometry (floor plan). In this study candles were used to observe slightest air movement across the room area. The room area is 10′ wide×16′ long×9′ high. An EFA was placed in different spots of the room and a deviation or flutter of a candle flame was assessed roughly by percentage of the maximum airflow which is directly in front of the EFA.

For instance, FIG. 7A shows the EFA located in the middle of the room with air flow direction toward the window. In this particular placement rather strong air flow (assessed by the candle's flame deviation) was observed in front of the EFA. Very weak flame deviation was seen along the long wall. In two other spots the candle flame, was essentially still. This analysis demonstrates that this particular EFA placement does not provide good air flow throughout the room and cannot ventilate and, therefore, clean air in all the areas.

In FIGS. 7(B, C, G and H) air flow is far from uniform either. All these settings can not be successful used for the complete air circulation and treatment in the room and cannot provide adequate air cleaning and disinfection.

The location shown in the FIG. 7D provides a better air flow with the EFA located in the middle of the room but fails to stir air along the walls. This is not ideal placement either.

Locations shown in the FIGS. 7E and 7J provide better solution but fail to stir air in certain areas either.

A quite different picture was observed when the EFA was placed near the window (in this case, with a curtain covering the window) and blowing toward the opposite direction, i.e. from the window to the flat wall with no objects near it (FIG. 7I). This location provides good air circulation as evidenced by visible disturbances of the candle plane throughout and in all corners of the room.

There are several reasons behind this phenomenon.

First, the surface towards which the EFA blows air should act to efficiently reflect the air back toward the EFA direction. The best surface for this purpose is a flat clean surface, such as a typical room wall. The window curtain or a blinds instead absorb air impacting those surfaces, deflect air flow in different directions and weaken the air stream. As a result return air flow is substantially slower and, in some portions of the room, is slower than required to provide good air stirring and ventilation.

Secondly, air flow generated by an EFA is rather linear, i.e. “beam-like” as opposed to the air flow generated by conventional fans that produce a “fan-like” air flow pattern. Therefore, the direct EFA air flow as well as any resultant reflected air flow tends to propagate in or along “narrow” channels. Therefore, optimal results may be achieved when air is directed along the longest room dimension as opposed being directed to the shortest room dimension.

Third, since an EFA generates a rather weak air flow, the best result is achieved by direct air flow as opposed to a reflected (even by the flat surface) air flow.

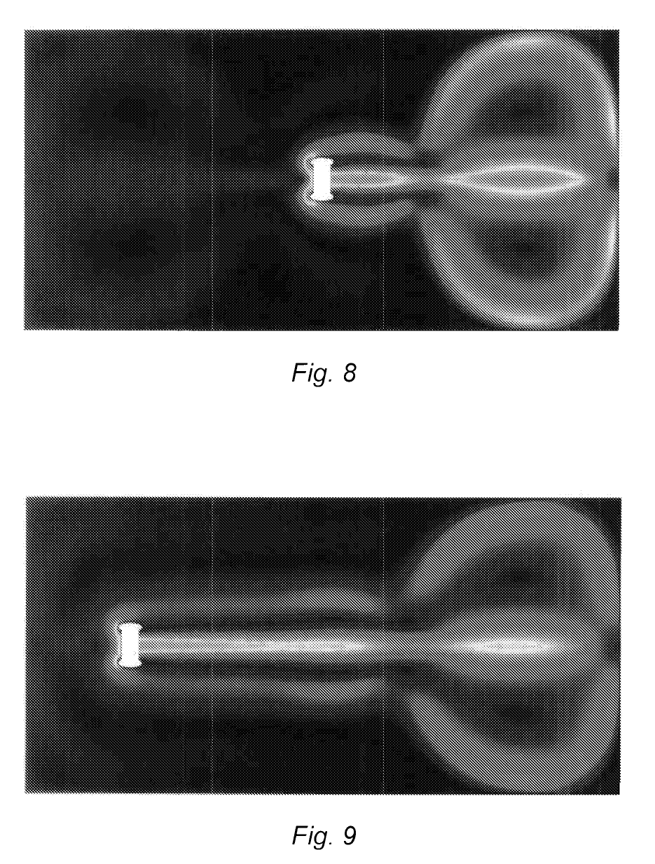

All the above reasons are illustrated in the FIGS. 8 trough 17. In these figures a computer modeling of a real EFA air flow is shown. Numerical results were obtained using COMSOL software, which is a commercial software package that performs equation-based multiphysics modeling for different physical processes by applying the finite element method to the system of partial differential equations. FIGS. 8-17 show two-dimensional airflow in an empty rectangular room produced by one or multiple EFA(s) placed at different locations. The direction of air velocity is shown by arrows while color represents air velocity magnitude from zero (dark blue) to highest magnitude (red).

In the FIG. 8 an EFA is installed in the middle of the room and is directed to blow air toward a flat wall. Air flow is rather strong in just one half of the room and rather weak in the half of the room that is behind the EFA.

In the FIG. 9 the EFA is moved closer to the short wall. Air flow is more uniform in the most part of the room. This location may be successfully used for the almost all room area ventilation and disinfection.

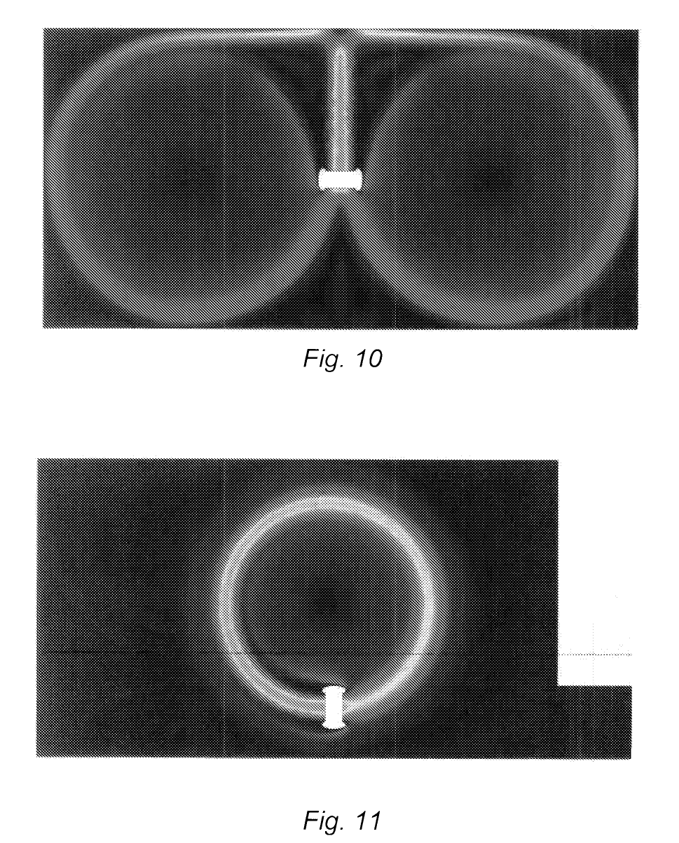

In the FIG. 10 EFA is located in the center of the room and it is directed to the long wall. Air flow is rather strong in the middle of the room and along the walls perimeter. This location does not provide good ventilation in all the room area. Some stagnant area may be observed in the right and left half-centers. These areas may be potentially dangerous in the presence of contagious people who introduce pathogens in the air.

As illustration, one principle of the air cleaning and disinfecting prefers all EFA orientation in which the EFA is located in such a manner that ensures the longest (available for the room) uninterrupted air path before meeting flat surface.

Another principle is as follows: the first surface onto which the air flow falls should reflect air back to the EFA inlet with the greatest speed away from the incident air flow, i.e. avoid deflecting the air in multiple weak streams so as to maximize the volume of air circulating. Ideally the initial air flow should be reflected into the area not affected by this initial air flow.

FIG. 11 shows another EFA location wherein the EFA is placed at the long wall and blows air along that wall. It is observed that air flow captures only a small portion of the entire room leaving much if not most of the air stagnant.

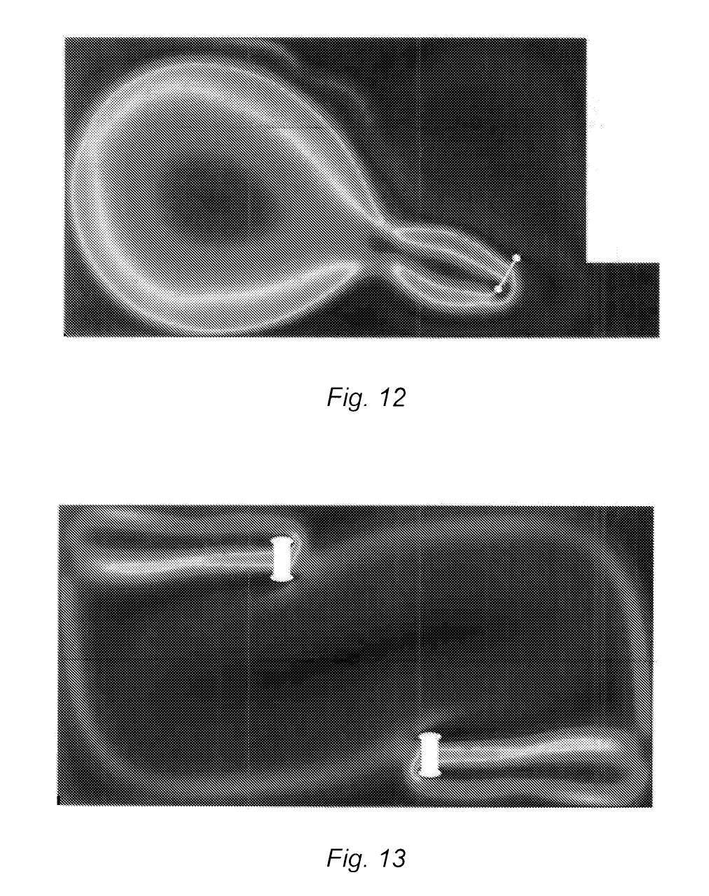

FIG. 12 shows still another EFA location where EFA is placed diagonally with respect to the room walls. This location is not efficient either as large areas of the room are outside the air flow.

More advantages may come out of the exceptional EFA ability to disinfect air and at same time to move it in straight, beam-like direction. If two or more EFA are placed in the same room in such a manner that they combine the forces applied to the air in the room, then greater portion of the air will be stirred and returned to the EFA inlet.

FIG. 13 shown “good” or an optimal multiple EFAs placement in the room. Two EFAs are located in such manner that air from each of them reflects from the wall and goes to the inlet of the other or next EFA. Passing through each EFA the air is cleaned and disinfected. It is rather clear from the shown air pattern that most of the air in the room goes through the air cleaning device thus ensuring good and swift air disinfection in the whole area.

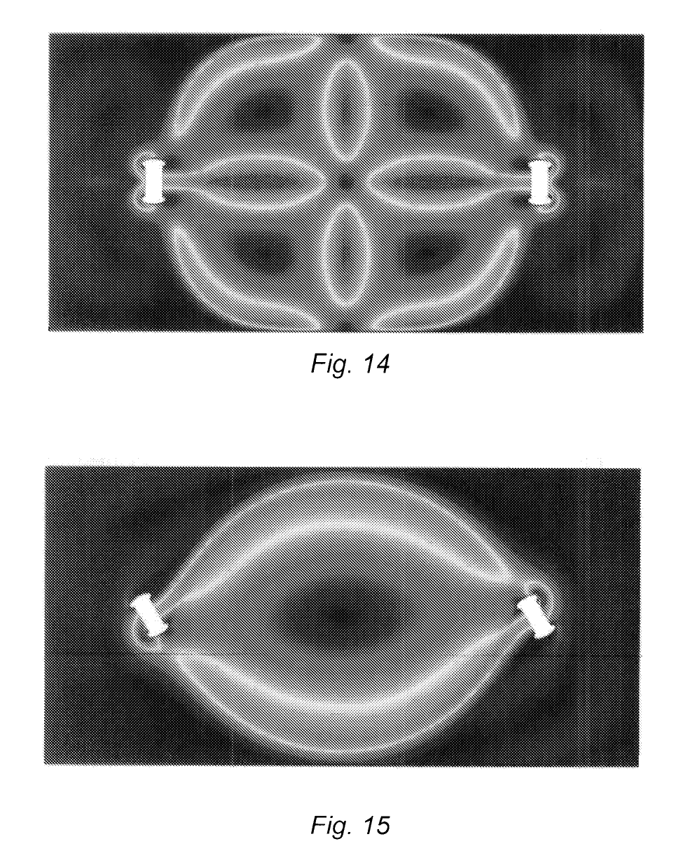

FIG. 14 shows a “bad” or less than optimal air pattern leaving substantial dead spots in the room. Herein, the two EFAs are located against each other and blow in opposite directions. Air is shown circulating with rather high speed in the room center. What is more important is that the air pattern is split into four vortexes and just a small amount of this air goes trough the EFAs inlet for the cleaning.

FIG. 15 shows still another “bad” configuration using two EFAs positioned at less than optimal locations. Air circulates in this case in the manner similar to the FIG. 14, i.e. most of it does not go through the EFA cleaning devices.

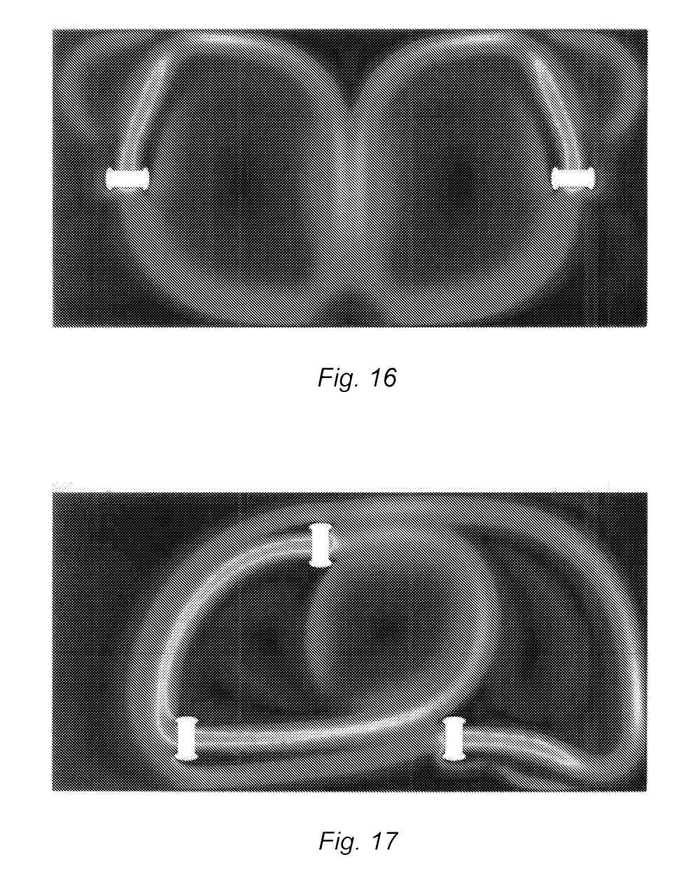

FIGS. 16 and 17 show another “good” multiple EFAs configuration. Air circulates here in such a manner that it goes to the EFAs inlet and is purified.

Other multiple EFA placement should be also considered in such manner that each of them delivers air straight to the inlet of the other or via reflection by or bouncing of an efficient barrier such as a room wall and other such structure or object.

On the other hand room objects like a large pieces of furniture (chests for instance) may be placed in such a manner that they provide a uniform air circulation through the most important or critical areas of a room.

It should be noted that EFA is not the only mean for the air acceleration and cleaning/disinfection. Air may be accelerated by the fans, ventilation and heat sources like batteries, fireplaces, heat exchangers and such. Further, air may be cleaned and disinfected by alternative and/or additional devices augmenting the cleaning process such as ultraviolet lamps, HEPA filters and by other means. Cleaning and disinfection of the air can also be improved by mixing the air inside a room using an EFA or by other means such as fans. Further, the means for air acceleration and the air cleaning/disinfection may be separate or (as in EFA case) combined in one.

As one skilled in the art will appreciate, air trajectories are the result of air acceleration and air reflection from walls, furniture and other objects. Such objects should be located in such a manner that most of the air is directed to the cleaning devices inlets. Most of the air to be cleaned should pass through the areas of major concern or to the areas of major concern. If the area of major concern is a bed with an infected patient then the air should pass this bed and them go to the cleaning device inlet. If the area of major concern is a nurse station or visitors place, clean air should go through air cleaning device to this area.

Although the invention has been described in connection with various illustrated embodiments, numerous modifications and adaptations may be made thereto without departing from the spirit and scope of the invention. Furthermore, it should be noted and understood that all publications, patents and patent applications mentioned in this specification are indicative of the level of skill in the art to which the invention pertains. All publications, patents and patent applications are herein incorporated by reference to the same extent as if each individual publication, patent or patent application was specifically and individually indicated to be incorporated by reference in its entirety.

1. The method of air cleaning and disinfecting, that includes
air acceleration by a first means;
air cleaning and/or disinfection by passing through a second means;
air delivery to an inlet of the second means by the first means.

2. The method of air cleaning and disinfecting according to the claim 1, further including a third means for providing an air pass configuration that ensures the most of the air is routed to go to said inlet of the second means.

3. The method of air cleaning and disinfecting according to the claim 1, that includes an air pass configuration by the first means that ensures a maximum amount of the air goes to said inlet of the second means.

4. The method of air cleaning and disinfecting according to claim 1, wherein at least a portion of the first means is combined with some portion of the second means.

5. The method of air cleaning and disinfecting according to claim 1, wherein air pass is configured in such a way that the air passes through areas of major conceal on the way to said inlet of the second means.

6. The method of air cleaning and disinfecting according to claim 1, wherein air pass is configured in such a way that air passes to areas of major concern on the way from an outlet of the second means

7. The method of air cleaning and disinfecting according to the claim 4, wherein an air pass is configured in such a way that it passes through areas of major concern on the way from an outlet of said first means to an inlet of said first means.

8. The method of air cleaning and disinfecting, according to the claim 2, wherein air is directed by said first means substantially orthogonally to a most distant third means.

9. The method of air cleaning and disinfecting, according to the claim 8, wherein an air pass from the first means to the third means is the longest available in the premises to be cleaned.

10. The method of air cleaning and disinfecting, according to the claim 1, wherein an air pass from an outlet of the first means to the inlet of said second means is shorter than an air pass from an outlet of the first means to an outlet of said second means.

11. The method of air cleaning and disinfecting, according to the claim 1, wherein the first means includes air acceleration by a heat source.

12. The method of air cleaning and disinfecting, according to the claim 1, wherein the first means include air acceleration by an existing ventilation and/or air exchange means.

13. The method of air cleaning and disinfecting, according to the claim 11, wherein the heat source includes one or more of the following: thermal batteries, space haters, fireplaces, heat exchanges and such.

14. The method of air cleaning and disinfecting, according to the claim 8, wherein air is reflected from the third means in substantially symmetrical fashion.

15. The method according to the claim 1, wherein air acceleration rates by said third means change with time.

16. A system for processing room air comprising:
an air propelling device located within the room, said air propelling device having an air intake operating to receive ambient room air, an air accelerator for accelerating the room air received by the intake, and an air exhaust operating to expel back into the room air from the air accelerator;
an air cleaning device operating to remove material from the room air; and
an air delivery device for routing the room air from said air cleaning device to said air intake of said air propelling device.

17. The system according to claim 16 wherein said air propelling device comprises an electrostatic fluid accelerator.

18. The system according to claim 16 wherein said air propelling device comprises an electric blower or fan.

19. The system according to claim 16 wherein said air cleaning device operates to disinfect the room air.

20. The system according to claim 16 wherein said air cleaning device comprises one or more of an air filter, ultraviolet lamp and electrostatic precipitator.

21. The system according to claim 16 further comprising an air deflector for directing a stream of air from the outlet of the air propelling device to the air cleaning device and ensuring that a major portion of air expelled by said air propelling device arrives at said air cleaning device.

22. The system according to claim 21 wherein said air deflector comprises a structural portion of the room.

23. The system according to claim 22 wherein said structural portion of the room comprises a wall of the room.

24. The system according to claim 21 wherein said air deflector is located at an opposite portion of the room across from the air propelling device.
